(19) United States
(12) Patent Application Publication
(10) Pub. No.: US 2007/0027195 A1
(43) Pub. Date: Feb. 1, 2007

(54) Novel compositions comprising higher primary alcohols and nicottinic acid and process of preparation thereof

(75) Inventors: Rajesh Jain, New Delhi (IN); Kour Chand Jindal, New Delhi (IN); Sukhjeet Singh, New Delhi (IN)

Correspondence Address: LADAS & PARRY, 26 WEST 61ST STREET, NEW YORK, NY, 10023, US

(73) Assignee: PANACEA BIOTEC LTD.

(21) Appl. No.: 11/489,254

(22) Filed: Jul. 19, 2006

Related U.S. Application Data
(63) Continuation-in-part of application No. PCT/IN05/00023, filed on Jan. 19, 2005.

(30) Foreign Application Priority Data
Jan. 20, 2004 (IN) 101/DEL/2004

(51) Int. Cl. A61K 31/455; A61K 31/045; A61K 47/00

(52) U.S. Cl. 514/355; 514/724; 514/772

(57) ABSTRACT

A composition comprising a mixture of higher primary aliphatic alcohols from 24 to 39 carbon atoms; at least one another component selected from resins and pigments, hydrocarbons, esters, ketones and aldehydes, and phenolic compounds, and nicotinic acid, its salts or derivatives thereof optionally with excipients, and process of preparation of such composition is provided. Also provided are method of treatment and use of such composition for reducing abnormal lipid parameters associated with hyperlipidemia. The compositions of the present invention are useful pharmaceutically or as a dietary supplement.

### CROSS REFERENCE

This application is a continuation-in-part of International Application No. IN2005/000023 filed on Jan. 19, 2005, which designated the U.S., claims the benefit thereof and incorporates the same by reference.

### FIELD OF THE INVENTION

The present invention relates to novel composition comprising a mixture of higher primary aliphatic alcohols from 24 to 39 carbon atoms; at least one another organic component selected from resins and pigments, hydrocarbons, esters, ketones and aldehydes, and phenolic compounds, and nicotinic acid, its salts or derivatives thereof optionally with excipients, and process of preparation of such composition. Also described are method of treatment and use of such composition thereof for reducing abnormal lipid parameters associated with hyperlipidemia. The compositions of the present invention are useful pharmaceutically or as a dietary supplement. Particularly, the present invention relates to compositions and method for lowering total cholesterol and triglycerides (TGs) level or elevating high density lipoprotein cholesterol (HDL-C) level in blood of a mammal.

### BACKGROUND OF THE INVENTION

Elevated serum cholesterol levels (>200 mg/dl) have been indicated as a major risk factor for heart disease, the leading cause of death worldwide. Atherosclerotic vascular diseases, and especially coronary heart disease (CHD), are the major cause of morbidity and mortality in middle age and elderly people worldwide (Pyorala et al., 1994; Sans et al., 1997). Thus, primary and secondary prevention of morbidity and death from CHD represents a major healthcare problem.

However, the use of currently available statins and fibrates should be used with caution in special patient population with increased susceptibility to drug-related adverse effects and frequent consumption of several concomitant medications, such as the elderly, patients with active hepatic diseases, etc. Furthermore, these lipid-lowering drugs are associated with adverse effects such as gastrointestinal disturbances, increase in serum transaminases, and creatinine kinase, myopathies, headache, cholelithiasis, impairment of fertility, and diminished libido. Due to the fact that cholesterol-lowering drugs must be administered on a long-term basis, there is still need of new effective and well-tolerated hypocholesterolemic agents.

Plant derived long-chain aliphatic alcohols have also been documented to reduce serum cholesterol levels in experimental models, and in type II hypercholesterolemic patients.

In the past few years, mixture of higher primary aliphatic alcohols has shown much promise, as reported in a number of published human clinical trials. The mechanism of action of such mixtures is not known, but various studies revealed that such mixtures inhibit cholesterol biosynthesis, increase the number of LDL-C receptors (Menendez et al., 1994) thereby decreases serum TC, LDL-C and increase HDL levels.

The mechanism of action of mixture of higher primary aliphatic alcohols is not known, but in vitro studies revealed that such mixture of higher primary aliphatic alcohols inhibit cholesterol biosynthesis at a step located in between acetate consumption and mevalonate production. In addition, in vitro studies also showed that the mixture of higher primary aliphatic alcohols increase the number of LDL-C receptors (Menendez et al., 1994). This accounts for the ability of such mixture not only to decrease total cholesterol, but also to decrease LDL serum levels and increase HDL levels. In vivo studies in correlation with in vitro studies demonstrated that such mixture inhibited TC and LDL-C induced by atherogenic diet suggesting possible inhibition of cholesterol biosynthesis (Menendez et al., 1996). In addition, administration of such mixture to diabetic patients significantly reduced TC and LDL-C levels in the blood (Omayda Torres et al., 1995).

U.S. Pat. No. 5,856,316 discloses a process for obtaining mixture of higher primary aliphatic alcohols from sugarcane wax and their utilization in the treatment of hypercholesterolemia. Such mixture from sugarcane wax comprise a mixture of aliphatic alcohols from 24 to 34 carbon atoms and they were effective hypocholesterolemic agents administered in daily doses from 1 to 100 mg.

U.S. Publication No. 20030232796 describes a composition comprising particles of at least one polycosanol or a salt thereof, wherein the polycosanol particles have an effective average particle size of less than about 2000 nm; and at least one surface stabilizer preferably selected from the group consisting of an anionic surface stabilizer, a cationic surface stabilizer, a zwitterionic surface stabilizer, and an ionic surface stabilizer.

Nicotinic acid, also known as niacin, has been used for many years in the treatment of hyperlipidemia. This compound has long been known to exhibit the beneficial effects of reducing TC, LDL-C, TGs, and Lp(a) in the human body, while increasing desirable HDL-C. Nicotinic acid, its salts or derivatives thereof has normally been administered three times per day after meals to provide a very beneficial effect on blood lipids as discussed in Knopp et al. (Metabolism 34, 645 (1985)). Though, such a dosing regimen produce beneficial effects, cutaneous flushing and the like often occurs in the hyperlipidemic patients to whom the compound is administered.

In order to avoid or reduce the cutaneous flushing, a number of materials have been suggested for administration with an effective antihyperlipidemic amount of nicotinic acid, including guar gum in U.S. Pat. No. 4,965,252, and mineral salts as disclosed in U.S. Pat. No. 5,023,245; or inorganic magnesium salts as reported in U.S. Pat. No. 4,911,917. These materials have been reported to avoid or reduce the cutaneous flushing side effect commonly associated with nicotinic acid, its salts or derivatives thereof treatment. Another method of avoiding or reducing the side effects associated with immediate release nicotinic acid is the use of sustained release formulations. Sustained release formulations are designed to slowly release the compound from the dosage form. The slow drug release reduces and prolongs blood levels of drug and thus minimizes the side effects. Sustained release formulations of nicotinic acid have been developed, such as Nicobid® capsules (Rhone-Poulenc Rorer) and Endur-acin® (Innovite Corporation).

U.S. Pat. No. 5,126,145 describes a sustained release nicotinic acid tablet formulation wherein the tablet comprises a hydroxypropyl methylcellulose having sustaining action, binder and a hydrophilic component. However, sustained release nicotinic acid formulations have experienced limited and restricted utilization because of the associated risk of potential liver damage.

The Patent Application No WO 0390547 relates to compositions comprising a waxy acid component consisting of at least a waxy acid with 23 to 50 carbon atoms and/or derivatives thereof and 0 to 99.99% by weight of at least a component with serum cholesterol level effecting properties and 0 to 20% by weight of at least a pharmaceutically acceptable formulation aid.

Therefore, it can be seen from the scientific literature that there is still an unmet need for development of new drugs or combinations of existing antihyperlipidemic agents with possible additive, potentiating or synergistic action preferably leading to dose reduction, and a method of administration which would provide a balanced lipid alteration i.e. reductions in TC, LDL-C, TGs, and Lp(a) as well as increases in HDL-C, with an acceptable safety profile, especially with regards to liver toxicity and effects on glucose metabolism and uric acid levels in hyperlipidemic patients.

### SUMMARY OF THE INVENTION

It is an objective of the present invention to provide novel composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and nicotinic acid, its salts or derivatives thereof substantially devoid of any waxy acid, optionally with excipients from 0 to 99.9% by weight of the composition.

It is an objective of the present invention to provide a process for preparing such composition which comprises of the following steps:
i) isolating the wax,
ii) subjecting the wax to extraction with a liquid organic extractant in which primary aliphatic alcohols and other organic components are soluble,
iii) recovering said soluble mixture from said extractant,
iv) purifying the extract by repeated washing and crystallization,
v) drying the extract and making it into a powder form,
vi) adding nicotinic acid, its salts or derivatives,
vii) optionally adding excipients and making it into a suitable dosage form.

It is yet another objective of the present invention to provide a method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and nicotinic acid, its salts or derivatives thereof, substantially devoid of any waxy acid, optionally with excipients from 0 to 99.9% by weight of the composition.

The compositions of the present invention have preferably a synergistic effect for reducing serum cholesterol level in mammals.

### DETAILED DESCRIPTION OF THE INVENTION

The present invention relates to novel composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and nicotinic acid, its salts or derivatives thereof.

The compositions of the present invention are substantially devoid of any waxy acid, optionally with pharmaceutically acceptable excipients from 0 to 99.9% by weight of the composition.

The mixture of higher primary aliphatic alcohols in the present invention are selected from but not limited to a group comprising 1-tetracosanol, 1-hexacosanol, 1-heptacosanol, 1-octacosanol, 1-nonacosanol, 1-tetratriacontanol, 1-triacontanol, 1-hexacontanol, eicosanol, 1-hexacosanol, 1-tetracosanol, 1-dotriacontanol, 1-tetracontanol, and the like. Preferably the mixture of higher primary aliphatic alcohols comprises 1-tetracosanol, 1-hexacosanol, 1-heptacosanol, 1-octacosanol, and 1-triacontanol.

In a further embodiment, the present invention provides a composition, wherein the mixture of higher primary aliphatic alcohols from 24 to 39 carbon atoms comprising 1-tetracosanol, 1-hexacosanol, 1-heptacosanol, 1-octacosanol, and 1-triacontanol are present as at least 40% by weight of the composition.

In a further embodiment, the present invention provides a composition, wherein the ratio of the mixture of higher primary aliphatic alcohols and nicotinic acid, its salts or derivatives thereof is from 20:1 to 1:20.

In another embodiment of the present invention, the mixture of higher primary aliphatic alcohols from 24 to 39 carbon atoms and the other organic component(s) selected from resins and pigments, hydrocarbons, esters, ketones and aldehydes, and phenolic compounds comprises of the following:

| | |
|---|---|
| 1-tetracosanol | 0.0-2.0% |
| 1-hexacosanol | 0.2-2.0% |
| 1-heptacosanol | 0.0-1.0% |
| 1-octacosanol | 30.0-40.0% |
| 1-triacontanol | 6.0-9.5% |
| Resins and pigments | 5.0-10.0% |
| Hydrocarbons | 1.0-10.0% |
| Esters | 1.0-10.0% |
| Ketones and Aldehydes | 1.0-10.0% |
| Phenolic compounds | 0.0-5.0% |

In a still further embodiment of the present invention, the mixture of higher primary aliphatic alcohols from 24 to 39 carbon atoms and the other organic component(s) selected from resins and pigments, hydrocarbons, esters, ketones and aldehydes, phytosterols, and phenolic compounds comprises of the following:

| | |
|---|---|
| 1-tetracosanol | 0.0-2.0% |
| 1-hexacosanol | 0.2-2.0% |
| 1-heptacosanol | 0.0-1.0% |
| 1-octacosanol | 30.0-40.0% |
| 1-triacontanol | 6.0-9.5% |
| Phytosterols | 0.1-1.0% |
| Resins and pigments | 5.0-10.0% |
| Hydrocarbons | 1.0-10.0% |
| Esters | 1.0-10.0% |
| Ketones and Aldehydes | 1.0-10.0% |
| Phenolic compounds | 0.0-5.0% |

The mixture of high-molecular weight aliphatic alcohols of the present invention occur naturally in wax form and are characterized by fatty alcohol chains ranging from 20 to 39 carbon atoms in length. The major components of such mixture are the aliphatic alcohols 1-octacosanol and 1-triacontanol, and the component includes 1-tetracosanol, 1-hexacosanol, 1-heptacosanol, 1-octacosanol, 1-nonacosanol, 1-tetratriacontanol, 1-triacontanol, 1-hexacontanol, eicosanol, 1-hexacosanol, 1-tetracosanol, 1-dotriacontanol, 1-tetracontanol, and the like; and other organic components such as resins and pigments, hydrocarbons, esters, ketones and aldehydes, phytosterols, phenolic compounds, and the like. Such mixture of high-molecular weight aliphatic alcohols and other organic components of the present invention are preferably isolated from a number of different sources, including sugar cane wax, beeswax, and rice bran wax, more preferably sugar cane wax. It should be understood, however, that the invention is not limited in this regard and that such mixture of high-molecular weight aliphatic alcohols commonly available from other naturally occurring and synthetic sources may be utilized.

The present invention employs nicotinic acid, its salts or derivatives thereof, or a compound other than nicotinic acid that the body metabolizes into nicotinic acid, thus producing the same effect as described herein. The other compounds specifically include, but are not limited to the following: nicotinyl alcohol tartrate, d-glucitol hexanicotinate, aluminum nicotinate, niceritrol and d, 1-alpha-tocopheryl nicotinate. Each such compound will be collectively referred to herein below by “nicotinic acid.” Nicotinic acid has multiple effects on lipoprotein metabolism. In adipose tissue, niacin inhibits the lipolysis of TGs by hormone-sensitive lipase, which reduces the transport of the free fatty acids to the liver and decreases hepatic TGs synthesis (Grundy et al., 1981). In addition it also reduces TG synthesis by inhibiting both the synthesis and esterification of fatty acids, effects that increase apoB degradation in the liver (Jin et al., 1999). Moreover, the reduction of TG synthesis further reduces hepatic VLDL production, which accounts for the reduced LDL levels. It also enhances lipoprotein lipids activity, which promotes the clearance of chylomicrons and VLDL triglycerides besides raising HDL-C levels by decreasing the clearance of apoA-I in HDL rather than by enhancing HDL synthesis (Blum et al., 1977). Further, it reduces the hepatic clearance of HDL-apoA-I, but not of cholesteroyl esters, thereby increasing the apoA-I content of plasma, augmenting reverse cholesterol transport (Jin et al., 1997).

The claimed mixture of high-molecular weight aliphatic alcohols and nicotinic acid, its salts or derivatives thereof, lower serum cholesterol links by two independent and unrelated mechanisms of action. The said mixture of high-molecular weight aliphatic alcohols inhibits cholesterol biosynthesis and increase the number of LDL-C receptors in liver. Nicotinic acid, its salts or derivatives thereof, acts by multiple mechanisms on lipid metabolism in liver and adipose tissue. However, these two compounds when combined showed a significant synergistic effect in lowering serum cholesterol. Thus, the combination of said mixture of high-molecular weight aliphatic alcohols and nicotinic acid, its salts or derivatives thereof into a single composition in the present invention provides a more effective treatment for elevated serum cholesterol than would be expected from the additive effect of both.

In an embodiment, the present invention provides dietary or pharmaceutical compositions suitable for lowering LDL-C and TGs level or elevating HDL-C level in blood of a mammal or both, by incorporating a combination of the mixture of high-molecular weight aliphatic alcohols, and at least one another organic component selected from resins and pigments, hydrocarbons, esters, ketones and aldehydes, and phenolic compounds; with nicotinic acid, its salts or derivatives thereof, derivatives, or mixtures thereof into some suitable food substance such as table margarine, shortenings, ice cream, yogurt and others or in pharmaceutical forms such as tablets or capsules or both which may also comprise a pharmaceutically acceptable excipient such as coloring agent, antioxidant, binder, stabilizer, and the like.

The present invention provides process for preparation of a fixed dose combination comprising of the mixture of high-molecular weight aliphatic alcohols, and at least one another organic component selected from resins and pigments, hydrocarbons, esters, ketones and aldehydes, and phenolic compounds; with nicotinic acid, its salts or derivatives thereof, derivatives, or mixtures thereof, optionally with excipient(s), which can be formulated as oral dosage forms such as tablets, pills, capsules, gels, finely divided powders, dispersions, suspensions, solutions, emulsions, etc; pulmonary and nasal dosage form such as sprays, aerosols, etc.; topical dosage forms such as gels, ointments, creams, etc; parenteral dosage forms; controlled release formulations; fast melt formulations, lyophilized formulations, delayed release formulations, sustained release, extended release formulations, pulsatile release formulations, and mixed immediate release and controlled release formulations.

The compositions of the present invention can be formulated for administration by the route selected from the group consisting of oral, pulmonary, rectal, colonic, parenteral, local, buccal, nasal, and topical.

In an embodiment of the present invention, the compositions can be preferably incorporated into compositions in the form of capsules. These capsules may also comprise excipients such as diluent, antioxidant, coloring agent, stabilizer, and the like. Composition can also be provided in the form of tablets comprising combination of the mixture of high-molecular weight aliphatic alcohols, and at least one another organic component selected from resins and pigments, hydrocarbons, esters, ketones and aldehydes, and phenolic compounds with nicotinic acid, its salts or derivatives or mixtures thereof, which may also comprise excipient(s) such as diluent, coloring agent, antioxidant, binder, stabilizer, and the like.

In an embodiment of the present invention, the composition as tablets/capsules or any other suitable pharmaceutical form are meant for lowering LDL-C level or elevating HDL-C level in mammals.

In an embodiment of the present invention, the ratio of the mixture of higher primary aliphatic alcohols or esters thereof and nicotinic acid, its salts or derivatives thereof is from 20:1 to 1:20.

In a further embodiment, the composition comprising a combination of a mixture of higher primary aliphatic alcohols from 24 to 39 carbon atoms comprising 1-tetracosanol, 1-hexacosanol, 1-heptacosanol, 1-octacosanol, and 1-triacontanol; phytosterols; resins and pigments; hydrocarbons; esters; ketones and aldehydes; and phenolic compounds with nicotinic acid, its salts or derivatives thereof, optionally comprises pharmaceutically acceptable excipients.

In a further embodiment, the pharmaceutically acceptable excipients are selected from but not limited to a group comprising diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, chelating agents and the like; used either alone or in combination thereof.

In the present invention, the diluent is selected from but not limited to a group comprising lactose, cellulose, microcrystalline cellulose, mannitol, dicalcium phosphate, pregelatinized starch, and the like, used either alone or in combination thereof.

In the present invention, the binder is selected from but not limited to a group comprising polyvinylpyrrolidone, cellulose derivatives such as hydroxypropyl methylcellulose, methacrylic acid polymers, acrylic acid polymers, and the like.

In the present invention, the release controlling polymer is selected from but not limited to a group comprising polyvinylpyrrolidone/polyvinylacetate copolymer (Kollidon® SR), methacrylic acid polymers, acrylic acid polymers, cellulose derivative, and the like. The methacrylic acid polymer is selected from a group comprising but not limited to Eudragit® (Degussa) such as Ammonio Methacrylate Copolymer type A USP (Eudragit® RL), Ammonio Methacrylate Copolymer type B USP (Eudragit® RS), Eudragit® RSPO, Eudragit® RLPO, and Eudragit® RS30D.

In an embodiment, the lubricant(s) used in the present invention are selected from, but not limited to a group comprising of stearic acid, magnesium stearate, zinc stearate, glyceryl behenate, cetostearyl alcohol, hydrogenated vegetable oil, and the like used either alone or in combination thereof.

In a further embodiment, the pharmaceutically acceptable excipients are present in about 0.5-80.0% by weight of the composition.

In a further embodiment of the present invention, a process for preparing a composition which comprises of the following steps:
i) isolating the wax,
ii) subjecting the wax to extraction with a liquid organic extractant in which primary aliphatic alcohols and other organic components are soluble,
iii) recovering said soluble mixture from said extractant,
iv) purifying the extract by repeated washing and crystallization,
v) drying the extract preferably at temperature below 70° C. and making it into a powder form,
vi) adding nicotinic acid, its salts or derivatives,
vii) optionally adding excipients and making it into a suitable dosage form.

The wax is preferably isolated from a number of different sources, including sugar cane, bees, and rice bran, more preferably sugar cane.

The liquid organic extractant of the present invention are selected from but not limited to a group comprising hexane, heptane, petroleum ether, chlorinated hydrocarbons, methanol, ethanol, isopropyl alcohol, ethyl acetate, acetone, ethyl methyl ketone, and the like, or mixtures thereof.

In the said process, the soluble mixture from the said extractant is recovered by distillation, with or without the application of vacuum.

The extract is purified preferably by repeated washing and crystallization. The solvents used for washing are selected from but not limited to hexane, heptane, petroleum ether, methanol, ethanol, isopropyl alcohol, ethyl acetate, acetone, ethyl methyl ketone, and the like, or mixtures thereof and the solvents for crystallization are selected from but not limited to hexane, heptane, petroleum ether, chlorinated hydrocarbons, methanol, ethanol, isopropyl alcohol, ethyl acetate, acetone, ethyl methyl ketone, toluene, and the like, or mixtures thereof.

The extract is dried by subjecting it to hot air oven, or by a Fluid bed drier, preferably at temperature below 70° C.

The present invention also provides a method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins and pigments, hydrocarbons, esters, ketones and aldehydes, and phenolic compounds from 0.1 to 70% by weight of the composition, and nicotinic acid, its salts or derivatives thereof, substantially devoid of any waxy acid, optionally with excipients from 0 to 99.9% by weight of the composition.

The compositions of the present invention have preferably a synergistic effect for reducing serum cholesterol level, and treating hyperlipidemia, particularly in mammals.

The ability of the mixture of higher primary aliphatic alcohols to inhibit cholesterol synthesis and of nicotinic acid, its salts or derivatives thereof to decrease total cholesterol (TC), low density lipoprotein cholesterol (LDL-C), TGs, and lipoprotein (a) (Lp(a)) while increasing HDL-C; when combined in the present invention results in preferably a synergistic effect in lowering serum cholesterol.

In an embodiment, the compositions for lowering LDL-C level or elevating HDL-C level in blood of a mammal or both, comprise a mixture of higher primary aliphatic alcohols, and at least one another organic component selected from resins and pigments, hydrocarbons, esters, ketones and aldehydes, and phenolic compounds; with nicotinic acid, its salts or derivatives thereof, and a method for lowering LDL-C and/or TGs level or elevating HDL-C level in blood of a mammal or both, comprises orally administering to said mammal, such compositions.

In an aspect of the present invention, the lipid lowering compositions comprising a mixture of higher primary aliphatic alcohols; at least one another organic component selected from resins and pigments, hydrocarbons, esters, ketones and aldehydes, and phenolic compounds; and nicotinic acid, its salts or derivatives thereof, derivatives, or mixtures thereof is associated with a reduction in the dose of nicotinic acid, its salts or derivatives thereof and increased patient compliance.

In the present invention, the mixture of higher primary aliphatic alcohols from 24 to 39 carbon atoms; and other organic components such as resins and pigments, hydrocarbons, esters, ketones and aldehydes, and phenolic compounds; is denoted as ‘Extract-A’.

### Determination of Biological Activity

### Cholesterol Diet-Induced Hypercholesterolemia in Mice

In the present invention, the observed unexpected synergistic lipid lowering effect of combination of Extract-A and nicotinic acid is evidenced by the test conducted in mice. Swiss mice of either sex were procured from Central Animal House facility; Panacea Biotec Ltd., India. Animals weighing 20-25 g at the time of testing were used throughout. All animals were dosed sequentially by the oral route with Extract-A and/or nicotinic acid suspended in 0.5% of carboxy methyl cellulose (CMC). A dosing volume of 10 ml/kg was used for each sequential suspension.

The fasting serum lipid profile (TC, TGs, LDL-C, HDL-C, and VLDL) was estimated before initiation of the experiment. Total study duration was of 8 weeks. Hypercholesterolemia was induced by feeding standard chow mixed with 1% cholesterol and 0.2% cholic acid to mice (Kaur and Kulkarni, 2000). Control animals received standard mice chow for 8 weeks whereas cholesterolemic control animals received hypercholesterolemic diet for 8 weeks. The presence of hyperlipidemia in mice was confirmed by estimating serum lipid parameters after 4 weeks. Thereafter, various doses of Extract-A and/or nicotinic acid were administered for another 4 weeks during which animals were fed with high cholesterol diet. Blood samples were collected from fasted mice and analyzed for any alteration in serum lipid profile after 4 weeks of test compound(s) administration.

All the data were expressed as mean ±S.E.M. (Standard Error of Mean). Student t-test was used to compare the lipid parameters between animals fed with standard and hypercholesterolemic diet. The difference between various drug treated groups was analyzed by ANOVA followed by Dunnett's test. A value of P<0.05 was considered as statistically significant.

Hypercholesterolemic diet for four weeks produced a significant alteration in all lipid parameters (TC, LDL-C, HDL-C, TGs and VLDL-C) in comparison to animal fed with standard mice chow. Extract-A dose dependently (10-40 mg/kg, p.o.) reversed TC and LDL-C levels in comparison to hypercholesterolemic control animals. Nicotinic acid dose dependently (10-40 mg/kg, p.o.) reversed all the serum lipid parameters in comparison to hypercholesterolemic control mice. Surprisingly, when lower doses of Extract-A (10 and 20 mg/kg) and Nicotinic acid (10 and 20 mg/kg) were administered in combination, a synergistic reduction in TC, TGs, VLDL-C, LDL-C, and increase in HDL-C levels was observed (Table 1 and FIGS. 1-5).

The data for the study is presented in Table -1, and shown diagrammatically in FIGS. 1-5.

### DESCRIPTION OF FIGURES

FIG. 1: Effect of Extract-A and/or nicotinic acid on serum total cholesterol in mice

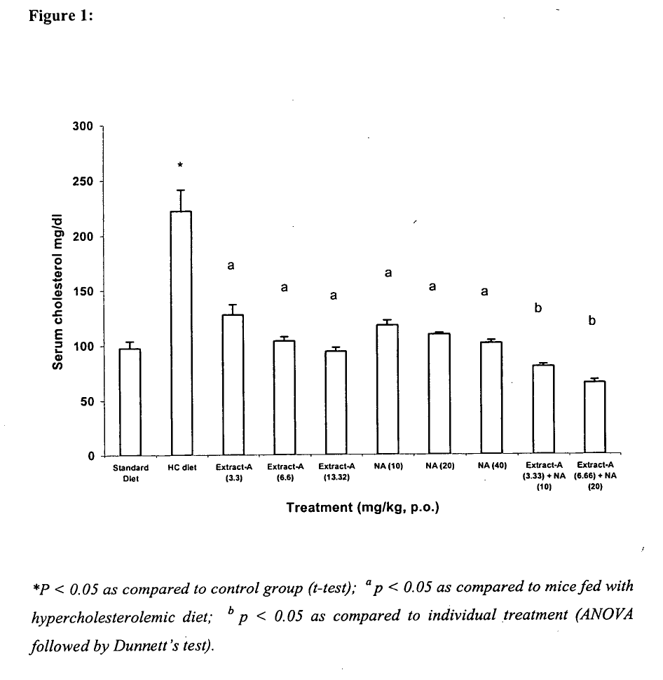

FIG. 2: Effect of Extract-A and/or nicotinic acid on serum triglycerides in mice

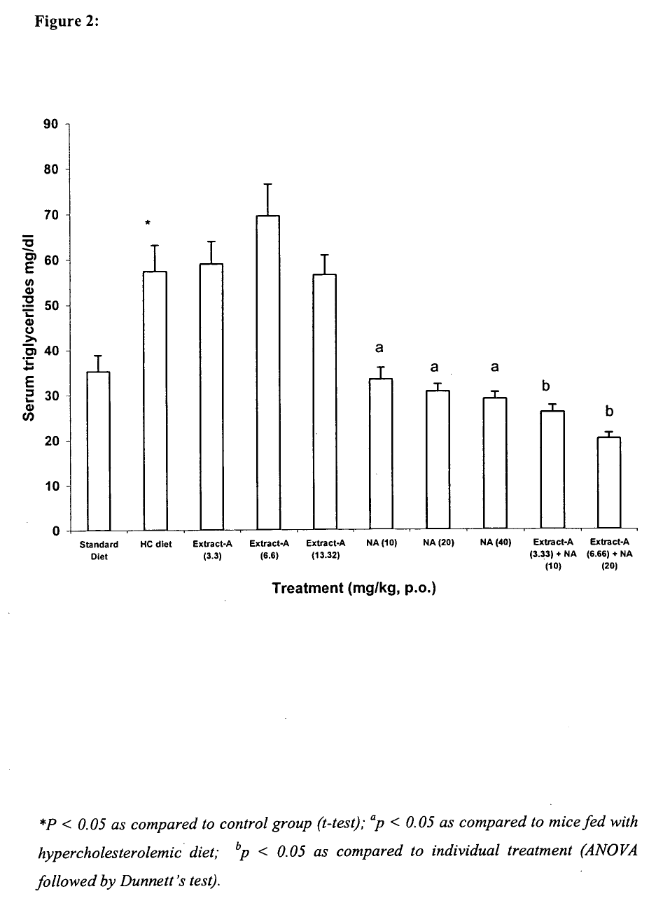

FIG. 3: Effect of Extract-A and/or nicotinic acid on HDL-C in mice

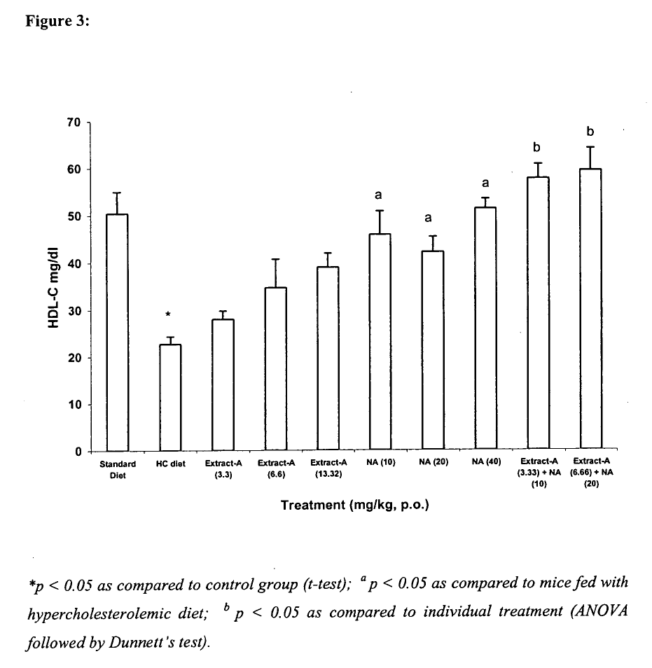

FIG. 4: Effect of Extract-A and/or nicotinic acid on LDL-C in mice

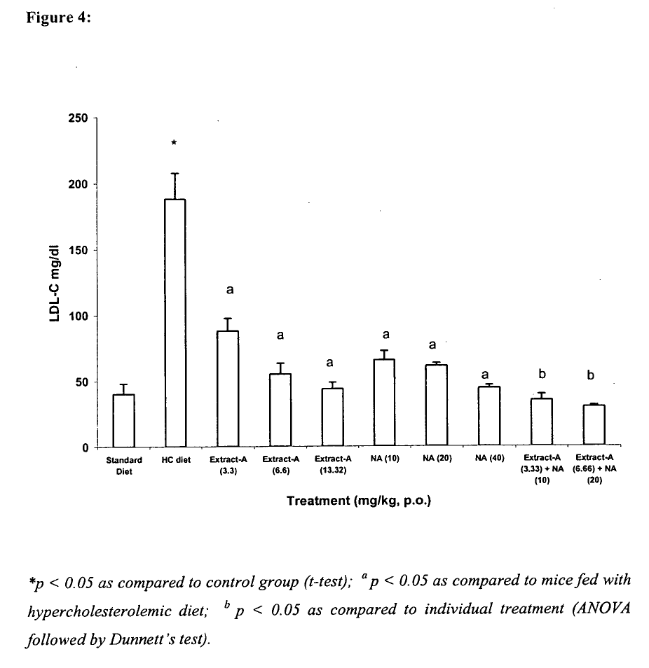

FIG. 5: Effect of Extract-A and/or nicotinic acid on VLDL-C in mice

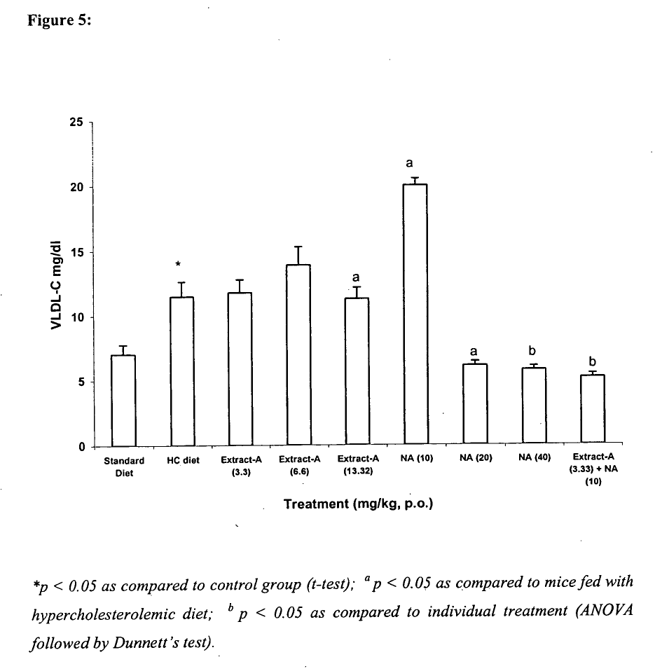

TABLE 1
Effect of policosanols and/or nicotinic acid, its salts or derivatives thereof on serum lipid profile in mice

| | | Total cholesterol | Triglycerides | | LDL-C | VLDL-C |
|---|---|---|---|---|---|---|
| Sr. No. | Treatment | mg/dl | mg/dl | HDL-C mg/dl | mg/dl | mg/dl |
| 1. | Standard diet | 97.62 ± 6.24 | 35.25 ± 3.63 | 50.37 ± 4.61 | 40.2 ± 7.98 | 7.05 ± 0.72 |
| 2. | Hypercholesterolemia diet | ^{222.33 ± 19.24*} | 57.41 ± 5.73* | 22.75 ± 1.59* | 188.1 ± 19.86* | 11.48 ± 1.14* |
| 3 | Extract-A (10 mg/kg) | 127.66 ± 9.45^{a} | 59.0 ± 4.95 | 28 ± 1.77 | 87.86 ± 9.60^{a} | 11.8 ± 0.99 |
| 4. | Extract-A (20 mg/kg) | 103.83 ± 3.84^{a} | 69.5 ± 7.0 | 34.66 ± 6.01 | 55.26 ± 8.15^{a} | 13.9 ± 1.40 |
| 5. | Extract-A (40 mg/kg) | 94.16 ± 3.78^{a} | 56.50 ± 4.31 | 39.0 ± 2.90 | 43.86 ± 4.98^{a} | 11.3 ± 0.86 |
| 6. | Nicotinic acid, its salts or | 118.0 ± 4.3^{a} | 33.3 ± 2.6^{a} | 45.82 ± 4.9^{a} | 65.5 ± 7.01^{a} | 20 ± 0.52^{a} |
| | derivatives thereof (10 mg/kg) | | | | | |
| 7. | Nicotinic acid, its salts or | 109.5 ± 1.60^{a} | 30.66 ± 1.58^{a} | 42.16 ± 3.19^{a} | 61.2 ± 2.14^{a} | 6.13 ± 0.31^{a} |
| | derivatives thereof (20 mg/kg) | | | | | |
| 8. | Nicotinic acid, its salts or | 101.6 ± 2.59^{a} | 29.0 ± 1.46^{a} | 51.33 ± 2.04^{a} | 44.53 ± 2.17^{a} | 5.8 ± 0.29^{a} |
| | derivatives thereof (40 mg/kg) | | | | | |
| 9. | Extract-A/nicotinic acid, its | 80.33 ± 2.17^{b} | 26 ± 1.57^{b} | 57.66 ± 3.0^{b} | 35.25 ± 4.44^{b} | 5.2 ± 0.30^{b} |
| | salts or derivatives thereof | | | | | |
| | (10/10 mg/kg) | | | | | |
| 10. | Extract-A/nicotinic acid, its | 65.33 ± 2.77^{b} | 20.16 ± 1.24^{b} | 59.33 ± 4.7^{b} | 30.04 ± 1.29^{b} | 4.03 ± 0.24^{b} |
| | salts or derivatives thereof | | | | | |
| | (20/20 mg/kg) | | | | | |

All the above values are expressed as mean ± S.E.M.
n (no. of mice) = 6-9 per group;
*p < 0.05 as compared to control group (t-test);
^{a}p < 0.05 as compared to mice fed with hypercholesterolemic diet;
^{b}p < 0.05 as compared to individual treatment (ANOVA followed by Dunnett's test).

The examples given below serve to illustrate embodiments of the present invention. However they do not intend to limit the scope of present invention.

### EXAMPLES

### Preparation of Extract

### Example 1

4 kg of air-dried Sugar mill Filter cake (or Press Mud) obtained as a byproduct during sugar manufacture from sugarcane was pulverized and extracted four times by boiling with 20 L of dichloroethane each time. The dichloroethane extract was filtered and the solvent was distilled off to get a dark green residue (400 g). The residue was extracted with 4 L of boiling methanol 3 times and the extract was filtered to remove the pitch while still hot (temperature above 50° C.). The filtered extract was distilled to remove methanol till a green residue (200 g) is obtained. The residue was dissolved in 2 L of boiling ethyl methyl ketone and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (50 g).

### Example 2

Beeswax obtained after extraction of honey from honeycomb was dried and pulverized and extracted four times by boiling with of ethyl alcohol each time. The alcoholic extract was filtered and the solvent was distilled off to get a residue. The residue was extracted with boiling methanol 3 times and the extract was filtered to remove the pitch while still hot (temperature above 50° C.). The filtered extract was distilled to remove methanol till a green residue is obtained. The residue was dissolved in boiling ethyl acetate and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant lumps were pulverized to a fine powder.

### Example 3

4 kg of air-dried Sugar mill Filter cake (or Press Mud) was pulverized and extracted four times by boiling with 20 L of hexane each time. The hexane extract was filtered and the solvent was distilled off to get a dark green residue (350 g). The residue was extracted with 3.5 L of boiling methanol 3 times and the extract was filtered to remove the pitch while still hot (temperature above 50° C.). The filtered extract was distilled to remove methanol till a green residue (200 g) is obtained. The residue was dissolved in 2 L of boiling acetone and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (45 g).

### Example 4

10 kg of air-dried Sugar mill Filter cake (or Press Mud) was pulverized and extracted four times by boiling with 50 L of methanol each time. The methanol extract was filtered and the solvent was distilled off to get a dark green residue (650 g). The residue was extracted with 6.5 L of boiling methanol 3 times and the extract was filtered to remove the pitch while still hot (temperature above 50° C.). The filtered extract was distilled to remove methanol till a green residue (500 g) is obtained. The residue was dissolved in 2 L of boiling ethyl acetate and set aside for crystallization. After complete crystallization the solvent is filtered, concentrated to half its volume by distillation and set aside for crystallization of the second crop. Both the crops were pooled and washed with cold hexane. The crystallization and washing procedures were repeated once more. The final washed crystals were dried under a current of air at a temperature not exceeding 70° C. The resultant creamish yellow lumps were pulverized to a fine powder (102 g).

### Preparation of Compositions

### Example 5 (Capsule)

| Ingredient | mg/capsule |
|---|---|
| 1. Extract-A | 80.0 |
| 2. Nicotinic acid | 500.0 |
| 3. Microcrystalline cellulose | 200.8 |
| 4. Mannitol | 72.0 |
| 5. Talc | 3.2 |
| 6. Sodium starch glycollate | 12.0 |
| 7. Colloidal silicon dioxide | 12.0 |

Procedure:
i) Extract-A, nicotinic acid, microcrystalline cellulose and mannitol are sifted and mixed together.
ii) Talc, sodium starch glycollate and colloidal silicon dioxide are passed through fine sieves individually and then mixed together.
iii) The materials of step (i) and (ii) are mixed and filled into empty hard gelatin capsules

### Example 6 (Uncoated Tablet)

| Ingredient | mg/tablet |
|---|---|
| 1. Extract-A | 80.0 |
| 2. Nicotinic acid | 500.0 |
| 3. Microcrystalline cellulose | 120.0 |
| 4. Mannitol | 80.0 |
| 5. Croscarmellose sodium | 10.0 |
| 6. Lactose | 66.0 |
| 7. Talc | 4.0 |
| 8. Colloidal silicon dioxide | 10.0 |
| 9. Croscarmellose sodium | 10.0 |

Procedure:
i) Extract-A, nicotinic acid, microcrystalline cellulose, mannitol, croscarmellose sodium and lactose are sifted and mixed together.
ii) The material of step (i) is compacted.
iii) The compacts of step (ii) are passed through sieve and mixed.
iv) Talc, colloidal silicon dioxide and croscarmellose sodium are passed through fine sieve and mixed together.
v) The material of step (iii) is mixed with material of step (iv).
vi) The material of step (v) is compressed into tablets.

### Example 7 (Film-Coated Tablet)

| | |
|---|---|
| Ingredient | mg/tablet |

Core tablet composition

| | |
|---|---|
| 1. Extract-A | 100.0 |
| 2. Nicotinic acid | 500.0 |
| 3. Microcrystalline cellulose | 120.0 |
| 4. Mannitol | 80.0 |
| 5. Croscarmellose sodium | 10.0 |
| 6. Lactose | 66.0 |
| 7. Talc | 4.0 |
| 8. Colloidal silicon dioxide | 10.0 |
| 9. Croscarmellose sodium | 10.0 |

Film coating composition

| | |
|---|---|
| 10. Hydroxypropyl methylcellulose (E-15) | 12.0 |
| 11. Polyethylene glycol 400 (PEG 400) | 2.4 |
| 12. Iron oxide red | 0.75 |
| 13. Iron oxide yellow | 0.50 |
| 14. Titanium dioxide | 0.25 |
| 15. Isopropyl alcohol | q.s. (lost in processing) |
| 16. Dichloromethane | q.s. (lost in processing) |

Procedure:
i) Extract-A, nicotinic acid, microcrystalline cellulose, mannitol, croscarmellose sodium and lactose are sifted and mixed together.
ii) The material of step (i) is compacted.
iii) The compacts of step (ii) are passed through sieve and mixed.
iv) Talc, colloidal silicon dioxide and croscarmellose sodium are passed through fine sieve and mixed together.
v) The material of step (iii) is mixed with material of step (iv).
vi) The material of step (v) is compressed into tablets.
vii) Hydroxypropyl methylcellulose is dispersed in a mixture of isopropyl alcohol and dichloromethane with continuous mixing in homogenizer.
viii) PEG 400 is added to the above solution of step (vii) and mixed.
ix) Iron oxide red, iron oxide yellow and titanium dioxide are passed through fine sieve and mixed.
x) The material of step (ix) is added to material of step (viii) and mixed for 30 minutes.
xi) The core tablets are charged into the coating pan and coated with the coating solution of step (x) till an average tablet weight gain of ˜2-3% is achieved.

### Example 8 (Bilayer Tablet)

A. Preparation of the Extract-A Layer

| Ingredients | mg/tablet |
|---|---|
| 1. Extract-A | 80.0 |
| 2. Microcrystalline cellulose | 120.0 |
| 3. Mannitol | 80.0 |
| 4. Croscarmellose sodium | 10.0 |
| 5. Lactose | 66.0 |
| 6. Talc | 4.0 |
| 7. Colloidal silicon dioxide | 10.0 |
| 8. Croscarmellose sodium | 10.0 |

Procedure:
i) Extract-A, microcrystalline cellulose, mannitol, croscarmellose sodium and lactose are sifted and mixed together.
ii) The material of step (i) is compacted.
iii) The compacts of step (ii) are passed through sieve and mixed.
iv) Talc, colloidal silicon dioxide and croscarmellose sodium are passed through fine sieve and mixed together.
v) The material of step (iii) is mixed with material of step (iv).

B. Preparation of the Nicotinic Acid, its Salts or Derivatives Thereof Layer

| Ingredients | mg/tablet |
|---|---|
| 1. Nicotinic acid | 500.00 |
| 2. Lactose | 85.00 |
| 3. Methacrylic acid copolymer | 60.00 |
| (Eudragit RSPO) | |
| 4. Stearic acid | 20.00 |
| 5. Isopropyl alcohol (IPA) | q.s. |
| 6. Dichloromethane | q.s. |
| 7. Magnesium stearate | 10.00 |
| 8. Stearic acid | 20.00 |

Procedure:
i) Mix Nicotinic acid, Lactose and Eudragit RSPO (40 mg) and pass through mesh size 40.
ii) Dissolve Eudragit RSPO (20 mg) and Stearic acid in IPA and Dichloromethane.
iii) Granulate the material of step (i) with the material of step (ii) and dry the granules.
iv) After drying the granules, pass them through a sieve of mesh size 60.
v) Pass Magnesium stearate and Stearic acid through sieve of mesh size 40 and mix with the dried granules.
C. Preparation of Bilayer Tablet

Compress the material of Step (v) of A with the material of step (v) of B into bilayer tablets.

We claim:

1. A composition comprising a mixture of higher primary aliphatic alcohols from 24 to 39 carbon atoms from 2 to 99.9% by weight of the composition; at least one other organic component selected from resins, pigments, hydrocarbons, esters, ketones, aldehydes, and phenolic compounds from 0.1 to 70% by weight of the composition, and nicotinic acid, or salt, analog or derivative thereof; optionally with excipients from 0 to 80% by weight of the composition; wherein the composition is substantially devoid of any waxy acid.

2. The composition according to claim 1, wherein the mixture of higher primary aliphatic alcohols comprises 1-tetracosanol, 1-hexacosanol, 1-heptacosanol, 1-octacosanol, and 1-triacontanol.

3. The composition according to claim 1, wherein the mixture of higher primary aliphatic alcohols from 24 to 39 carbon atoms comprises 1-tetracosanol, 1-hexacosanol, 1-heptacosanol, 1-octacosanol, and 1-triacontanol and are present as at least 40% by weight of the composition.

4. The composition according to claim 1, wherein the ratio of the mixture of higher primary aliphatic alcohols and nicotinic acid or, a salt, analog or derivative thereof is from 20:1 to 1:20 weight/weight.

5. The composition according to claim 1, wherein the pharmaceutically acceptable excipients are selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents or a mixture thereof.

6. The composition according to claim 1, which is formulated in an oral; pulmonary; nasal; topical; parenteral; controlled release; fast melt; lyophilized; delayed release; sustained release; extended release; pulsatile release; mixed immediate release; or controlled dosage form.

7. The composition according to claim 6, wherein the oral dosage form is selected from the group consisting of a tablet, pill, capsule, gel, powder, dispersion, suspension, solution and emulsion.

8. The composition according to claim 6, wherein the nasal or pulmonary dosage form is a spray or aerosol.

9. The composition according to claim 6, wherein the topical dosage form is selected from the group consisting of a gel, ointment and cream.

10. A process for preparing a composition according to claim 1, which comprises the steps of:
i) isolating a wax,
ii) subjecting the wax to extraction with a liquid organic extractant in which primary aliphatic alcohols and other organic components are soluble,
iii) recovering said soluble mixture as an extract from said extractant,
iv) purifying the extract by repeated washing and crystallization,
v) drying the extract and making it into a powder form,
vi) adding nicotinic acid, a salt, analog or derivative thereof,
vii) optionally adding pharmaceutically acceptable excipients, and making it into a suitable dosage form.

11. The process according to claim 10, wherein the mixture of higher primary aliphatic alcohols comprises 1-tetracosanol, 1-hexacosanol, 1-heptacosanol, 1-octacosanol, and 1-triacontanol.

12. The process according to claim 10, wherein the mixture of higher primary aliphatic alcohols from 24 to 39 carbon atoms comprises 1-tetracosanol, 1-hexacosanol, 1-heptacosanol, 1-octacosanol, and 1-triacontanol are present as at least 40% by weight of the composition.

13. The process according to claim 10, wherein the ratio of the mixture of higher primary aliphatic alcohols and nicotinic acid, a salt, analog or derivative thereof is from 20:1 to 1:20 weight/weight.

14. A method of reducing serum cholesterol level, and treating hyperlipidemia, which comprises administering a composition comprising a mixture of higher primary aliphatic alcohols from 24 to 39 carbon atoms from 2 to 99.9% by weight of the composition; at least one another organic component selected from resins, pigments, hydrocarbons, esters, ketones, aldehydes, and phenolic compounds from 0.1 to 70% by weight of the composition, and nicotinic acid or a salt, analog or derivative thereof; optionally with excipients from 0 to 80% by weight of the composition; wherein the composition is substantially devoid of any waxy acid.
